(19) United States
(12) Patent Application Publication
(10) Pub. No.: US 2008/0228259 A1
(43) Pub. Date: Sep. 18, 2008

(54) ENDOVASCULAR DEVICES AND METHODS TO PROTECT ANEURYSMAL WALL

(76) Inventor: Jack Fa-De Chu, Santa Rosa, CA (US)

Correspondence Address: JACK CHU, 5918 SUNHAWK DRIVE, SANTA ROSA, CA, 95409, US

(21) Appl. No.: 12/046,196

(22) Filed: Mar. 11, 2008

Related U.S. Application Data
(60) Provisional application No. 60/895,232, filed on Mar. 16, 2007.

(51) Int. Cl. A61F 2/06

(52) U.S. Cl. 623/1.11

(57) ABSTRACT

Methods and systems for preventing aneurysm rupture and reducing the risk of migration and endoleak are disclosed. Specifically, a hardenable liner is applied directly to treat the interior of the aneurysm site. The hardening agent remains flexible in the pliable mode so that the liner can be collapsed and compressed in a delivery catheter. After deploying in the aneurysm, the flexible liner expands by the hemodynamic force and conforms to the inner surface of the aneurysm. While the liner is still conforming to the aneurysm, the hardening agent in the liner is activated by the body environment and becomes stiff. The liner is thus transformed from the pliable mode to the strengthening mode and providing support for the aneurysm wall. Also disclosed are methods to deliver the hardenable liner directly to treatment sites.

### FIELD OF THE INVENTION

Methods and devices for preventing rupture of an aneurysm and reducing the risk of endoleak are disclosed. Specifically, methods and systems for applying inflatable multiple-layer liners directly to treatment sites and to the interior of the vessel wall are provided. This application claims the benefit of U.S. Provisional Application No. 60/895,232, which was filed Mar. 16, 2007, the disclosure of which is incorporated herein by this reference.

### BACKGROUND OF THE INVENTION

An aneurysm is a localized dilation of a blood vessel wall usually caused by degeneration of the vessel wall. These weakened sections of vessel walls can rupture, causing an estimated 32,000 deaths in the United States each year. Additionally, deaths resulting from aneurysmal rupture are suspected of being underreported because sudden unexplained deaths are often misdiagnosed as heart attacks or strokes while many of them may in fact be due to ruptured aneurysms.

Approximately 50,000 patients with abdominal aortic aneurysms are treated in the U.S. each year, typically by replacing the diseased section of vessel with a tubular polymeric graft in an open surgical procedure. However, this procedure was risky and not suitable for all patients. Patients who were not candidates for this procedure remained untreated and thus at risk for aneurysm rupture or death.

A less-invasive procedure is to place a stent graft at the aneurysm site. Stent grafts are tubular devices with one or more metallic stents attached to the polymeric grafts such as Dacron® or ePTFE film. The size of the tubular graft is usually matched to the diameter of the healthy vessel adjacent to the aneurysm. The metallic stent is generally stitched, glued or molded onto the biocompatible tubular covering and provides strength to the graft. In other embodiments, one or more inflatable channels were attached to the tubular graft for additional strength, and, in some cases, replaced the metal scaffold. Usually, stent grafts can be positioned and deployed at the site of an aneurysm using minimally invasive procedures. Essentially, a delivery catheter having a tubular stent graft compressed and packed into the catheter's distal tip is advanced through an artery to the aneurismal site. The tubular stent graft is then deployed within the vessel lumen in juxtaposition to the diseased vessel wall, and forming a flow conduit without replacing the dilated section of the vessel. This new flow conduit insulates the aneurysm from the body's hemodynamic forces, therefore decreasing hemodynamic pressure on the disease portion of the vessel and reducing the possibility of aneurysm rupture.

While tubular stent grafts represent improvements over more invasive surgery procedures, there are still risks associated with their use to treat aneurysms. Stent graft migration and endoleak are the biggest challenges for tubular stent grafts due to several reasons. Frequently, most of the support for the tubular stent graft depends on its fixation on a very limited section of healthy vessel between the renal artery and the aneurysm, i.e. at the neck of the aneurysm. The aneurysm sac between the aneurysm wall and the tubular stent graft is usually filled with blood or unorganized thrombosis providing little or no support to the stent graft. This vulnerable aneurysm sac is also prone to endoleak. Stent graft migration is especially common in aneurysms with short neck where there is insufficient overlap between the stent graft and the vessel, and in tortuous portions of the vessels where stent graft tends to kink resulting high hemodynamic forces on the stent graft.

Stent graft migration can break the seal between the tubular stent graft and vessel and lead to Type I endoleak, or the leaking of blood into the aneurismal sac between the outer surface of the stent graft and the inner surface of the blood vessel. This endoleak can result in the aneurysm wall being exposed to hemodynamic pressure again, thus increasing the risk of rupture.

Other than Type I endoleak, many patients experience some other issues after undergoing stent graft therapy for their aneurysms. Type II endoleak is defined as the leakage due to patent collateral arteries in the aneurismal sac. The patent collateral arteries (inferior mesenteric artery, lumbar artery) in the aneurismal sac can lead to an increased pressure in the aneurysm and may cause aneurysm enlargement and rupture in some patients. Type III and IV endoleaks are leaks caused by defects in the stent grafts. As a result, physicians often have to follow up closely with patients after endovascular therapy and perform secondary intervention to stop the leakage if it is required. Both follow-up procedures and secondary interventions are undesirable because the cost and the risk involved in those procedures.

Based on the foregoing, one goal of treating aneurysms is to provide a therapy that does not migrate or leak. To achieve this goal, stent grafts with anchoring barbs or hooks that engage the vessel wall have been developed to enhance their attachment to the wall as described in U.S. Patents and patent applications U.S. Pat. Nos. 6,395,019B2, 7,081,129B2, 7,147,661B2, 2003/0216802A1. Additionally, endostaple that punches through both graft and vessel wall to fix stent graft to the vessel wall has been developed. While these physical anchoring devices have proven to be effective in some patients, tubular stent grafts are still prone to kink. Migration and endoleaks are still reported in many patients.

Other than the improvement of the stent graft, several attempts have been made to prevent endoleak by embolizing the aneurismal sac with thrombosis or fillers such as coils, gel, fibers, etc. U.S. Pat. Nos. 6,658,288 and 6,748,953 discussed the methods to use electrical potential to create thrombosis in the aneurysm. U.S. Patents and patent applications U.S. Pat. Nos. 5,785,679, 6,231,562, 6,613,037, 7,033,389, 637,973, 6,656,214, 633,100, 6,569,190, 2003/135264A1, 36,745A1, 44,358A1, 2005/90804A1 and WO95/08289 disclose methods and devices to embolize the aneurismal sac. Those methods and devices create hardened material in the aneurismal sac to prevent endoleaks. However, embolization agent or dislodged emboli can travel downstream and embolize small vessels in the legs or colon. As a result, a stent graft or a barrier layer is usually utilized to exclude the aneurismal sac from the major blood conduit before injecting embolization agent into the aneurismal sac. This approach reduces the chance for the emboli to pass through the barrier layer and travel to the iliac arteries. However, the junctions to the collateral vessels in the aneurismal sac are not protected. Physicians usually will occlude the patent collateral vessels before the embolization procedure. Unfortunately, it is very difficult to identify the patency of the collateral vessels (inferior mesenteric artery, lumbar artery) in the aneurismal sac by the current imaging techniques, such as CT or MRI. If those collateral vessels are patent, i.e. a Type II endoleak is diagnosed, there is a risk that the injected embolization agent or dislodged emboli will migrate into those collateral vessels and embolize important vessels in the lumbar and colon.

Due to the risk of accidental embolization, some have proposed that the injected filler is contained in a graft or a membrane and the aneurismal sac be isolated before the injection of filler, as disclosed in U.S. Patent and patent application U.S. Pat. Nos. 6,729,356, 5,843,160, 5,665,117, 2004/98096A1 and 2006/212112A1, which are fully incorporated by reference herein. The fill structure generally has a spherical shape, and there is typically a tubular main conduit in the middle for restoring the original geometry of the flow conduit. However, there are several concerns with this approach. First, to avoid endoleaks and migration, a close contact between the outer wall of the fill structure and the aneurysm wall is important to seal the junctions of the aorta to the origins of the collateral branch arteries. Because the fill structure is constrained by the aneurysm wall and the stent graft (or a shaping balloon) in the middle, it is essential to inject sufficient amount of filler in the fill structure to maintain close contact between the aneurysm wall and fill structure and, at the same time, avoid injecting excess amount of filler and exerting additional stress on the weak aneurysm wall. However, the gap between the fill structure and the aneurysm wall cannot be visualized easily (no contrast agent in gap or aneurysm wall) under Fluoroscope during the inflation of the fill structure, physician cannot determine if the gap has been filled (or not being filled) by the fill structure. This uncertainty can cause excess amount of filler in the fill structure and consequently high stress on the aneurysm wall and place the patient in great risk. Additionally, the aneurysm is usually sealed by a stent graft or a lumen shaping balloon before the fill structure is inflated. Existing blood in the aneurysm (with the added filler) can also cause high stress on the aneurysm wall during the inflation of fill structure if the collateral arteries in the aneurysm are occluded. Third, a significant amount of filler is required to fill the aneurismal sac for patients with large aneurysms. The effect of this large chunk of filler on vessel movement and the adjacent organs is still unknown.

Thus, there is a need to develop a new method to treat an aneurysm site to protect the aneurysm and reduce the risk of endoleak and rupture. The present invention addresses this opportunity by providing methods and systems to protect the aneurysm and to reduce the likelihood of endoleak, migration and rupture at aneurysm sites.

### SUMMARY OF THE INVENTION

The present invention addresses the issues with the current therapies by providing methods and systems to reduce the likelihood of migration, endoleak and rupture at aneurysm sites. The systems comprise a hardenable liner which is larger or the same size as the aneurysm. The hardenable liner comprises a hardening agent between two flexible walls. It has a pliable mode and a strengthening mode. In its pliable mode, this hardenable liner is flexible and can be loaded into a catheter. After the liner is introduced in the aneurysm, the liner expands and conforms to the surface of the aneurysm wall by a hemodynamic force. The inner surface of the liner defines the flow conduit. The environmental change such as temperature, moisture, pH, etc. activates the hardening agent in the liner and transforms the liner from the pliable mode to the strengthening mode. The resulting strengthened liner can protect the aneurysm wall and is “locked” in the aneurysm with minimum chance to migrate out of its designated location.

According to the teaching of this invention, many suitable hardening agents can be used for the hardenable liner. The preferable hardening agent is a non-biodegradable material such as acrylate or silicone polymer. It can be cured by the moisture and has been used as bone cement or implant for many years. A radiopaque agent such as barium sulfate or gold powder can also be included in the hardening agent or in the wall to enhance the liner's visibility under fluoroscopy and serve as a filler. At least one wall of the liner has to be permeable to the moisture in the body. After the liner is deployed in the aneurysm, the body fluid diffuses through the wall and reacts with the hardening agent. The hardening agent becomes rigid and thus stiffens the liner. After the liner is stiffened, it transforms into strengthening mode and is locked in the aneurysm providing reinforcement to the aneurysm wall.

In another embodiment of this invention, the hardening agent comprises calcium compound. Calcium phosphate cement (CPC) is biocompatible and has been used as bone cement and dental implant for years. The cement will harden when exposed to water and form hydroxyapatite (HA) which is a key component of human bone. A radiopaque agent such as barium sulfate or gold powder can be included in the calcium compound or in the wall to enhance the liner's visibility under fluoroscopy. The calcium compound can be laminated and encapsulated between the walls by spraying, coating, dipping, etc. At least one of the walls is permeable to the moisture in the aneurysm. After the calcium compound is encapsulated between the walls, the hardening reaction will not occur until it is exposed to water. The calcium compound paste remains flexible in the pliable mode so that the liner remains flexible and can be compressed and loaded in a delivery catheter. After the liner is deployed in the aneurysm, the body fluid diffuses through the wall and reacts with the calcium compound. The stiffened calcium compound in the liner strengthens the liner, and the liner is thus converted from the pliable mode to the strengthening mode providing support for the aneurysm wall.

In another embodiment of this invention, the hardening agent comprises a polymer solution. Those biocompatible polymers are preferable not to be degradable in the body. They should be soluble in biocompatible solvent but not in water. The exemplary non-limiting biocompatible solvents are ethanol, dimethylsulfoxide (DMSO), ethyl lactate, acetone, etc. The exemplary non-limiting biocompatible polymers are cellulose acetate, cellulose acetate butyrate, cellulose diacetate, nitrocellulose, polyurethane, polycarbonate, polyester, ethylene vinyl alcohol, ethylene vinyl acetate, polyacrylonitrile, polyvinyl acetate, polyvinyl alcohol, acrylics, and copolymer and mixture thereof. The polymer solution will precipitate and solidify when it is in contact with water and the solvent is leached out of the polymer solution. The polymer solution can be laminated between the walls by spraying, coating, dipping, etc. At least one of the walls is permeable to the water but not soluble to the solvent in the aneurysm. After the polymer solution is encapsulated between the walls, the hardening reaction will not occur until it is exposed to water. The polymer solution remains flexible in the pliable mode so that the liner remains flexible and can be compressed and loaded in a delivery catheter. After the liner is deployed in the aneurysm, the water diffuses through the wall and precipitates the polymer in the liner. The solidify polymer in the liner strengthens the liner, and the liner is thus converted from the pliable mode to the strengthening mode providing support for the aneurysm wall.

In another embodiment according to the present invention, a bioactive or a pharmaceutical agent is incorporated into the liner. The bioactive or pharmaceutical agent can be mixed with the hardening agent before laminating in the liner. After deploying in the aneurysm, the bioactive or pharmaceutical agent diffuses into the aneurysm wall and treats the damage in the vessel. Because the liner of this invention is in close contact with the aneurysm wall, the bioactive or pharmaceutical agent can reach the aneurysm wall without being diluted by the blood. Many bioactive or pharmaceutical agents can be used to treat aneurysm. Drugs that inhibit matrix metalloproteinases, inflammation or other pathological processes involved in aneurysm progression, can be incorporated into the filler to enhance wound healing and/or stabilize and possibly reverse the pathology. Drugs that induce positive effects at the aneurysm site, such as growth factor, can also be delivered with the hardening agent and the methods described herein. Alternatively, the bioactive or pharmaceutical agent can be coated on the outer surface of the hardenable liner directly against the sac wall.

In another embodiment of the present invention, the surface of the liner is treated with a fibril, coating, foam or surface texture enhancement. These coatings or surface treatment can increase the surface area on the outer wall of the liner and promote tissue or cell to grow onto the outer surface of the liner. The attached cells or tissue on the liner can enhance the bonding and seal between the vessel wall and the liner. In addition to enhanced bonding, appropriate surface coating or texture can also promote the formation of thrombosis and increase the seal between the liner and the aneurysm wall.

In another embodiment of this invention, a plurality of hardenable liners can be deployed sequentially in an aneurysm to increase their protection on the aneurysm. Several hardenable liners can be used in the same aneurysm to increase the total thickness of the liners. Alternatively, hardenable liners of different constructions can be used to achieve the optimum liner performance. For example, hardenable liner facing the aneurysm wall can comprise a more porous outer surface with a better tissue attachment to the aneurysm surface. On the other hand, hardenable liner facing the flow conduit can comprise more hardening agent with a better support to the flow conduit.

In another embodiment of the present invention, the hardenable liner is particularly suitable for lining aneurysm close to the bifurcation, especially abdominal aortic aneurysms (AAA) adjacent to the iliac bifurcation. The liner is hollow and flexible with three openings. Because the liner is larger or the same size as the aneurysm, the liner is expanded plastically by the hemodynamic force and conforms to the inner surface of the aneurysm after deploying in the aneurysm. The inner surface of the liner determines the blood flow conduit with one inlet and two outlets. After deployed in the aneurysm, the liner would have a shape defined by the inner surface of the aneurysm. The blood flow conduit would have a shape determined by the inner surface of the aneurysm and the thickness of the liner.

In another embodiment of the present invention, the hardenable liner is particularly suitable for lining aneurysm which has extended from aorta to the iliac artery. The liner is hollow and flexible with a bifurcation. Because the liner is larger or the same size as the aneurysm, the liner is expanded plastically by the hemodynamic force and conforms to the inner surface of the aneurysm after deploying in the aneurysm. The inner surface of the liner determines the blood flow conduit with one inlet and two outlets. The blood flow conduit would have a shape determined by the inner surface of the aneurysm and the thickness of the liner.

Other than the hardenable liner, the systems to treat aneurysm also include at least one stent which is placed near the opening of the liner after the liner is deployed in the aneurysm. Preferably, the stent is deployed at the junction between the liner and the vessel wall to ensure no gap between them. Usually, the stent is most useful to be deployed at the inlet of the blood conduit because the high hemodynamic shear force near the edge of the liner. Optionally, stent can be deployed at the outlet of the blood conduit. No stent will be needed in the middle of the aneurysm because the liner is already hardened, and the hemodynamic force will keep the liner against the inner surface of the aneurysm maintaining the patency of the flow conduit. Alternatively, portion of the stent can be covered with a graft or a membrane to further assist the sealing between the liner and vessel wall. Alternatively, a stent can be permanently attached to the liner near the opening of the liner.

In the practice, physician needs to determine the appropriate liner to use in each patient. Through the imaging techniques such as CT scan or MRI, the size and length of the patient's aneurysm can be measured accurately. Then, the physician can select a liner that best fit the patients' aneurismal anatomy. It is preferred to use a liner with outer diameter no less than the largest inner diameter of the aneurysm. Because the flexible walls of the liner and the hemodynamic force in the liner, the liner will remain conform to the inner surface of the aneurysm.

For a preferred deployment method of this invention, a multi-lumen catheter with an expandable element is used to deliver the hardenable liner in the aneurysm. The expandable element has a first configuration and a second configuration. The first configuration allows the expandable element to be compressed into the catheter for delivery with minimum invasivity. The second configuration allows the expandable element to expand and anchor the liner at the proximal end of the aneurysm. Additionally, the expandable element on the multi-lumen catheter is configured to allow blood perfusion through the expandable element at the second configuration. Many expandable elements, such as balloon, stent, etc. can be used in this invention. An annual shape balloon is used herein as an example. In its pliable mode, portion of the hardenable liner near the inlet is placed on top of the balloon with its inner surface against the balloon. After the liner and balloon are both collapsed into the low profile configurations, they can be compressed and loaded into a sheath on the catheter and sterilized with various known sterilization methods. Then, the liner delivery system can be positioned in the aneurysm site via iliac artery with minimum invasivity. It is preferable that the balloon on the distal end of the catheter is deployed near the neck of the aneurysm to ensure no excess stress is applied on the aneurysm. After the balloon is deployed, portion of the hardenable liner near the inlet is pressed against the vessel wall by the inflated balloon. At the same time, blood flows through the lumen in the balloon to expand the liner radially toward the aneurysm wall. As the sheath is retrieved to expose the liner in the sheath, the expansion continues until the liner covers the whole inner surface of the aneurysm. This procedure is safe because the pressure to expand the liner is the same pressure existed in the aneurysm before the operation. No additional stress is placed on the aneurysm wall during the expansion of the liner. As the liner is expanding, the status of expanding is monitored by the radiopaque markers on the liner. Alternatively, a radiopaque agent is incorporated into the liner so that the whole liner is visible under fluoroscope. After the inner surface of the aneurysm wall is completely covered by the liner, a second expandable element (e.g. balloon) is inflated at the other junction between the liner and the vessel. This second balloon can be on the same multi-lumen catheter or on a separate one. The purpose of this second balloon is to ensure the patency of flow conduit after the deployment of liner. The transition of the liner from pliable mode to the strengthening mode gives addition strength to the liner and protects the aneurysm. It is accomplished by activating the hardening agent in the liner through the environmental change from being in the catheter to being in the body. Because the liner is already conformed to the inner surface of the aneurysm, the transition “locks” the hardened liner in the aneurysm against the aneurysm wall. Then the balloons are collapsed, and the delivery catheter is retrieved from the patient's body. Optionally, a stent or a membrane covered stent is placed at junction between the liner and the vessel wall to ensure seal.

In another deployment method of this invention for treating patient with aneurysm close to the bifurcation (iliac artery), a multi-lumen catheter with an expandable element is used to deliver the liner in the aneurysm. The expandable element has a first configuration and a second configuration. The first configuration allows the expandable element to be compressed into the catheter for delivery with minimum invasivity. The second configuration allows the expandable element to expand and anchor the liner at the proximal end of the aneurysm. Additionally, the expandable element on the multi-lumen catheter is configured to allow blood perfusion through the expandable element at the second configuration. Many expandable elements, such as balloon, stent, etc. can be used in this invention. An annular balloon with flow lumen is described herein as an example. The balloon is positioned near the distal end of the multi-lumen catheter. In the collapsed configuration, portion of the liner nears the inlet is placed on top of the balloon with its inner surface against the balloon. After the liner and balloon are collapsed into low profile configurations, they are compressed and loaded into a sheath in the multi-lumen catheter and sterilized. Then, the catheter/liner system can be positioned in the aneurysm site via the iliac artery with minimum invasivity. It is preferred that the balloon is deployed near the neck of the aneurysm to ensure no excess stress is applied on the aneurysm. After the balloon is inflated, portion of the liner near the inlet is pressed against the vessel wall by the inflated balloon. Then, the sheath of the catheter is removed to expose the liner. As soon as the liner is exposed, it expands radially toward the aneurysm wall by the hemodynamic pressure through the lumen in the balloon and eventually conforms to the inner surface of the aneurysm wall. After the inner surface of the aneurysm wall is completely covered by the liner, a second expandable element (e.g. balloon) is inflated at the first junction between the liner and the iliac artery. This balloon can be on the same multi-lumen catheter or on a separate one. A third expandable element (e.g. balloon), which is on a different catheter, is deployed at the other iliac artery junction. The purpose of the second and the third balloons is to ensure the patency of the flow outlets after the expansion of liner. The transition of the liner from pliable mode to the strengthening mode gives addition strength to the liner and protects the aneurysm. It is accomplished by activating the hardening agent in the liner through the environmental change from being in the catheter to being in the body. Because the liner is already conformed to the inner surface of the aneurysm, the transition “locks” the liner in the aneurysm against the aneurysm wall. After the liner is hardened, the balloons are collapsed, and the delivery catheter is retrieved from the patient's body. Optionally, a stent or a membrane covered stent is placed at junction between the liner and the vessel wall to ensure seal.

There are several benefits to treat aneurysm with this present invention. 1. The hardenable liner strengthens the aneurysm wall and prevents the rupture of aneurysm by reducing the hemodynamic pressure on the aneurysm wall. 2. The collapsed liner is flexible so that it can be loaded in a catheter and access the aneurysm site with minimum invasivity. 3. The flexibility of the liner and the hemodynamic force allow the liner to conform to the inner surface of the aneurysm wall. After the liner is strengthened, it will be “locked” in the aneurysm without endoleak or migration. 4. Less material is required to cover the inner surface of the aneurysm wall. The resulting liner is more flexible and compatible with the vessel and adjacent organs. 5. There is no excess amount of stress on the vulnerable aneurysm wall during the deployment of the liner. In order to prevent endoleak and migration, it is essential to have close contact between the outer wall of the liner and the surface of the aneurysm wall. This invention addresses the drawbacks of prior arts and allows the liner to conform to the aneurysm wall without placing excess stress on the fragile aneurysm wall. As a result, the systems and methods provided by this present invention are safer than methods disclosed in prior arts. 6. The flexible liner does not have the issue of kinking or occlusion of blood flow which is common in tubular stent graft. 7. The durability of the liner is better than the tubular stent graft because there is no untreated space, which is prone to endoleak, between the liner and aneurysm wall. 8. The present invention can enhance the adhesion of the liner to the aneurysm wall further reducing the risk of liner migration and endoleak. 9. This invention enables the use of bioactive or pharmaceutical agents in the liner to treat aneurysm without dilution. The pathological processes involved in aneurysm progression can be stabilized and possibly be reversed.

### BRIEF DESCRIPTION OF THE FIGURES

FIG. 1a depicts the perspective view of a hardenable liner as described in one embodiment according to the present invention.

FIG. 1b depicts the cross sectional view of the hardenable liner as described in FIG. 1a.

FIG. 2 depicts the cross sectional view of a hardenable liner as described in one embodiment according to the present invention.

FIG. 3 depicts the cross sectional view of a hardenable liner as described in one embodiment according to the present invention.

FIG. 4a depicts the perspective view of a hardenable liner as described in one embodiment according to the present invention.

FIG. 4b depicts the cross sectional view of the hardenable liner as described in FIG. 4a.

FIG. 5a depict the perspective view of a hardenable liner as described in one embodiment according to the present invention.

FIG. 5b depicts the cross sectional view of the hardenable liner as described in FIG. 5a.

FIGS. 6a-e depict the perspective views of hardenable liners as described in one embodiment according to the present invention.

FIG. 7a depicts the exterior view of a delivery catheter as described in one embodiment according to the present invention.

FIG. 7b depicts the collapsed hardenable liner on a delivery catheter as described in one embodiment according to the present invention.

FIGS. 8a-h depict deploy sequences of a hardenable liner in the aneurysm according to the teachings of the present invention.

FIGS. 9a-h depict an alternate method to deploy a hardenable liner in the aneurysm according to the teachings of the present invention.

FIGS. 10a-i depict an alternate method to deploy a hardenable liner in the aneurysm according to the teachings of the present invention.

FIG. 11 depicts the cross sectional view of hardenable liners deployed in the aneurysm as described in one embodiment according to the present invention.

### DETAILED DESCRIPTION

Embodiments according to the present invention provide hardenable liners and methods useful for protecting aneurysm and reducing the risk of device post-implantation migration and endoleak. More specifically, the hardenable liners and methods address the root cause of the issue by providing protection to the aneurysm site directly against rupture. The hardenable liners also have the advantage of minimizing post-implantation device migration and post-implantation endoleak following liner deployment at an aneurismal site.

For convenience, the devices, compositions and related methods according to the present invention discussed herein will be exemplified by using hardenable liner intended to treat abdominal aorta aneurysm or Thoracic aortic aneurysm. However, aneurysms at other locations of the body can be treated with the same devices or methods.

The present invention addresses the issues with current therapies by providing methods and systems to reduce the likelihood of migration, endoleak and rupture at aneurysm sites. This system comprises a hardenable liner which is larger than or the same size as the aneurysm to be treated. The liner is hollow with an outer surface and an inner surface. The inner surface of the liner defines the blood flow conduit. This liner has both pliable mode and strengthening mode. In the pliable mode, this hardenable liner is flexible and can be compressed and loaded in a delivery catheter. Because the liner is larger than or the same size as the aneurysm, the liner expands under a hemodynamic force and conforms to the inner surface of the aneurysm without a gap. This close contact with aneurysm wall is important because it allows no vulnerable “gap” or “space” between the liner and the weak aneurysm wall that is prone to endoleak. After deploying in the aneurysm, the body environment such as temperature, moisture, pH, etc. activates the hardening agent in the liner. The liner changes from the pliable mode to the strengthening mode by the activated hardening agent resulting in a stronger liner to support the aneurysm wall. This body environment can be caused by the body itself or by chemical or physical means introduced in the aneurysm by a catheter. Because the hardened liner is conforming to the usually complex topography of the inner surface of the aneurysm, hardened liner is “locked” in the aneurysm without migrating out of its designated location and provides reinforcement to the weak aneurysm wall.

The close contact with aneurysm wall is important for the hardenable liner to eliminate any gap between the liner and the aneurysm wall. Other than the properties of the liner, the deploying method is important to achieve conformation to the aneurysm wall. The hemodynamic force to expand the liner has to be sufficient to expand the liner without causing excess stress on the aneurysm wall. Additionally, the existing blood in the aneurysm has to be drained from the aneurysm while the liner is expanding. The details of the deploying method will also be disclosed in this invention.

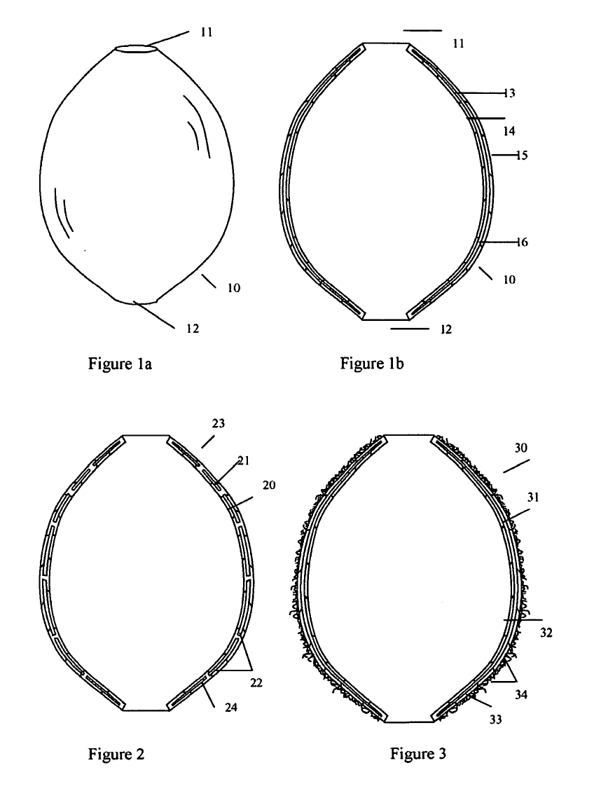

In the present invention, hardenable liner 10 has a general appearance of a hollow pouch with two openings 11, 12 as illustrated in the perspective view in FIG. 1a. The embodiment of this invention with two openings is particularly suitable for treating patients with Thoracic aortic aneurysm (TAA), aneurysms in the peripheral arteries, or abdominal aortic aneurysms (AAA) with some distance from the iliac bifurcation. As shown in the cross sectional view of hardenable liner 10 in FIG. 1b, hardenable liner 10 comprises a hardening agent 13 encapsulated by flexible inner wall 14 and outer wall 15. Walls 14, 15 are joined together at two openings 11, 12 and form chamber 16 filled by hardening agent 13. Before hardening agent 13 is activated and becomes rigid, hardening agent 13 is flexible and enhances the flexibility of liner 10. In its pliable mode, liner 10 is flexible and can be compressed and loaded in a delivery catheter. After the delivery catheter has reached its aneurysm site, the flexibility of liner 10 enables it to expand radially and conform to the aneurysm wall under a hemodynamic force. After liner 10 is conformed to the inner surface of the aneurysm, it is stiffened and transformed into the strengthening mode so that it can be “locked” in the aneurysm to prevent migration and provide reinforcement to the weak aneurysm wall. The transformation or stiffening of liner 10 is activated by the body environment or by a chemical or physical means introduced by a catheter in the aneurysm,

Walls 14, 15 of liner 10 serve as a means not only to contain hardening agent 13 but also control the timing for hardening agent 13 to activate and change liner 10 from the pliable mode to the strengthening mode. For example, if hardening agent 13 is activated by the moisture in the body after liner 10 is deployed, at least one of walls 14, 15 is permeable to the moisture and with permeability sufficient to allow enough moisture to penetrate through walls 14, 15 and activate hardening agent 13 within liner 10. Wall 14, 15 with low moisture permeability will delay the diffusion of moisture and increase the “operation window” to compete deploying liner 10 before it is hardened in the aneurysm. In addition, the thickness of walls 14, 15 is also important in determining the diffusion rate for the moisture to penetrate walls 14, 15 and activate hardening agent 13. Thicker wall 14, 15 delays the diffusion of moisture and increases the “operation window” to compete deploying liner 10 before it is hardened in the aneurysm. As a result, the hardening time (i.e. the time required to stiffen the hardening agent) in various area of liner 10 can be tailored according to the requirements. For example, a thinner or a high permeability wall 14, 15 can be used in area requires fast hardening such as the neck of the aneurysm (i.e. the healthy vessel near the proximal end of the aneurysm). Hardened liner 10 near the aneurysm neck can enhance the anchoring of liner 10 in the aneurysm. A thicker or a low permeability wall 14, 15 can be used in area requires a longer hardening time or a pliable state such as area near the bifurcation. This allows a longer “operation window” to compete deploying liner 10 before it is hardened in the aneurysm.

The materials used for walls 14, 15 are biocompatible and flexible so that walls 14, 15 can conform to the inner surface of the aneurysm. According to the teaching of the present invention, walls 14, 15 can be constructed with sheets or films. Each wall 14, 15 can be made from the same or a different biocompatible material. Typical biocompatible materials are Dacron®, Nylon, PET, PE, PP, polyurethane, ethylene vinyl acetate, FEP or ePTFE. They can be extruded, weaved, blow molded or molded into thin sheet or film. The processing technologies are well known to person specialized in film or sheet processing. The thin sheet or film is stitched, glued, bonded or directly molded into the desired shape. Or they can be made by spraying, coating, and dipping, etc. polymer solution directly on a mold and dried. Permeable wall can be fabricated by creating holes or pores on the wall by laser, by punctuation or by salt leaching process, etc. The techniques to manufacturing permeable wall are known to people in this art. Hardening agent 13 can be laminated between walls 14, 15 by spraying, coating, dipping, etc. Alternatively, as shown in FIG. 2, walls 20, 21 are joined in selected connections 22 to ensure the integrity of liner 23 before hardening agent 24 is activated. Because there is no hardening agent 24 at connections 22, liner 23 is not stiffened at connections 22 while it is in the strengthening mode. Those relatively “soft” connections 22 can also serve as a “stress relief” for hardened liner 23 in the aneurysm. They enable hardened liner 23 to remain conformation to the inner surface of the aneurysm while allowing some flexibility for body movement.

In another embodiment of the present invention, the outer surface of the liner is treated to increase its surface area. As illustrated in FIG. 3, hardenable liner 30 comprises hardening agent 31, inner wall 32, outer wall 33 and fibrils 34 on outer wall 33. The increased surface area by attached fibrils 34 can increase the contact between the vessel and liner 30. Due to the intimate contact with the outer surface of liner 30, smooth muscle cells and fibroblasts, etc. in the vessel will be stimulated to proliferate. As these cells proliferate they will grow onto the surface of liner 30 so that liner 30 becomes physically attached to the vessel lumen. The attached cells or tissue on liner 30 can enhance the bonding and seal between the vessel wall and liner 30. In addition, the increase surface area also promotes the formation of thrombosis. The thrombosis can fill any existing gap between the outer surface of liner 30 and the surface of the aneurysm wall further preventing endoleak. Additionally, the enhanced bonding between liner 30 and the aneurysm surface can seal the junctions of the collateral arteries in the aneurysm and prevent Type II endoleak. Typical techniques to increase surface area are sanding, etching, depositing, coating, and bonding with fibrils, a porous layer or thin foam. They are well known to the people skilled in the art.

According to the teaching of this invention, many suitable hardening agents can be used for hardenable liner. The preferable hardening agent is a non-biodegradable material such as a polymer, an oligomer or a monomer which can harden after being activated by the body environment or other physical or chemical means introduced by a catheter in the aneurysm. The hardening of the non-biodegradable material can be triggered by either physical or chemical means. Chemical means include curing, cross-linking, polymerization, etc. The hardening agent can be either one component or two components. Two components hardening agent usually comprises a resin and a curing agent. The physical means often involve change in temperature, light, radiation, electricity, pH, ionic strength, concentration, etc. However, because the relatively large size of the aortic aneurysm, it is difficult to radiate and cure the hardening agent in the liner with even dosage of radiation (e.g. UV light) if the hardening agent requires radiation curing. It will also be difficult to control the operation time required to complete the procedures before the pre-mixed two components hardening agent is hardened. The preferable hardening agent disclosed in present invention is an acrylate or a silicone polymer. It can be cured by the moisture and has been used as bone cement or implant for many years. Exemplary non-limiting moisture-cured hardening agents include acrylate, cyanoacrylate, isobutyl cyanoacrylate, n-butyl cyanoacrylate, silicone, their oligomers or monomers or mixture thereof. The polymerization initiators for the moisture-cured silicone can be selected from those well known to people in the art. The exemplary non-limiting common initiators are acetoxy, oxime, etc. Additional fillers such as poly(methyl methacrylate), fibers, particles are usually needed to increase the composition's viscosity for the ease of handling and its stiffness after curing. A radiopaque agent such as barium sulfate or gold powder can also be included in hardening agent or in wall to enhance the liner's visibility under fluoroscopy and serve as a filler. To use moisture cured resin as hardening agent, liner and delivery catheter have to remain dry before use. At least one wall of liner has to be permeable to the moisture in the body. After liner is deployed in the aneurysm, body fluid diffuses through wall and reacts with the resin to complete the curing. The cured resin becomes rigid and thus stiffens liner. After liner is stiffened, it transforms into strengthening mode and is locked in the aneurysm providing reinforcement to the aneurysm wall.

In another embodiment of this invention, the hardening agent comprises calcium compound. Calcium phosphate cement (CPC) is biocompatible and is consisting of dicalcium phosphate anhydrous(DCPA), CaCO3, Ca(OH)2, alpha-tricalcium phosphate(alpha-TCP), or tetracalcium phosphate (TTCP), etc. The cement will harden when exposed to water and form hydroxyapatite (HA) which is a key component of human bone. In addition to calcium phosphate, other calcium containing compounds can also be used as hardening agent in this invention. Exemplary non-limiting compounds are amorphous calcium phosphate, monocalcium phosphate monohydrate, monocalcium phosphate anhydrous, dicalcium phosphate dehydrate, dicalcium phosphate anhydrous, beta-tricalcium phosphate, octacalcium phosphate, and mixture thereof, etc. Other than calcium phosphate, sodium phosphate and organic acid such as carboxylic acid can be used to accelerate the hardening once the calcium compound is in contact with water. Additional thickening agent such as celluloses (MC, CMC, HMC, etc.), collagen, alginate, glycerol, polypropylene glycol, polyethylene glycol can also be used with calcium compounds to form a paste for the ease of handling. A radiopaque agent such as barium sulfate or gold powder can be included in the calcium compound or in the wall to enhance the liner's visibility under fluoroscopy. The calcium compound can be laminated and encapsulated between the walls by spraying, coating, dipping, etc. At least one of the walls is permeable to the moisture in the aneurysm. After the calcium compound is encapsulated between the walls, the hardening reaction will not occur until it is exposed to water. These flexible walls on the liner serve as a means not only to contain the calcium compound but also control the timing for the calcium compound to activate and change the liner from the pliable mode to the strengthening mode. The permeability of the wall is sufficient to allow enough moisture to penetrate through the walls and activate the calcium compound within the liner. The calcium compound paste remains flexible in the pliable mode so that the liner remains flexible and can be compressed and loaded in a delivery catheter. After the liner is deployed in the aneurysm, the body fluid diffuses through the wall and reacts with the calcium compound. The stiffened calcium compound in the liner strengthens the liner, and the liner is thus converted from the pliable mode to the strengthening mode providing support for the aneurysm wall. To use calcium compound as hardening agent, the liner and delivery catheter have to remain dry before use.

In yet another embodiment of this invention, the hardening agent comprises a polymer solution. Those biocompatible polymers are preferable not to be degradable in the body. They should be soluble in biocompatible solvent but not in water. The exemplary non-limiting biocompatible solvents are ethanol, dimethylsulfoxide (DMSO), ethyl lactate, acetone, etc. The exemplary non-limiting biocompatible polymers are cellulose acetate, cellulose acetate butyrate, cellulose diacetate, nitrocellulose, polyurethane, polycarbonate, polyester, ethylene vinyl alcohol, ethylene vinyl acetate, polyacrylonitrile, polyvinyl acetate, polyvinyl alcohol, acrylics, and copolymer and mixture thereof. A radiopaque agent such as barium sulfate, silver, or gold powder can also be included in the polymer solution or in the wall to enhance the liner's visibility under fluoroscopy. The polymer solution will precipitate and solidify when it is in contact with water and the solvent is leached out of the polymer solution. The polymer solution can be laminated between the walls by spraying, coating, dipping, etc. At least one of the walls is permeable to the water in the aneurysm. After the polymer solution is encapsulated between the walls, the hardening reaction will not occur until it is exposed to water. These flexible walls on the liner serve as a means not only to contain the polymer solution but also control the timing for the polymer solution to harden and change the liner from the pliable mode to the strengthening mode. The permeability of the wall is sufficient to allow enough water to penetrate through the walls and solidify the polymer within the liner. The polymer solution remains flexible in the pliable mode so that the liner remains flexible and can be compressed and loaded in a delivery catheter. After the liner is deployed in the aneurysm, the water diffuses through the wall and precipitates the polymer in the liner. The solidify polymer in the liner strengthens the liner, and the liner is thus converted from the pliable mode to the strengthening mode providing support for the aneurysm wall. To use polymer solution as hardening agent, the solvent should not dissolve the materials used for walls and delivery catheter.

In another embodiment according to the present invention, the liner comprises a bioactive or a pharmaceutical agent. The bioactive or pharmaceutical agent can be incorporated in the hardenable liner or mixed with hardening agent before encapsulating in the liner. After deploying in the aneurysm, the bioactive or pharmaceutical agent diffuses into the aneurysm wall and treats the disease in the vessel. Because the hardenable liner of this invention is in close contact with the aneurysm wall, the bioactive or pharmaceutical agent can reach the aneurysm wall without being diluted by the blood. Many bioactive or pharmaceutical agents can be incorporated in the hardenable liner to treat aneurysm in this invention. Agents that inhibit matrix metalloproteinases, inflammation or other pathological processes involved in aneurysm progression, can be incorporated into the hardenable liner to enhance wound healing, stabilize and possibly reverse the pathology of aneurysm. Agents that induce positive effects at the aneurysm site, such as growth factor, can also be delivered by the hardenable liner and the methods described herein. Exemplary non-limiting examples include platelet-derived growth factor (PDGF), platelet-derived epidermal growth factor (PDEGF), fibroblast growth factor (FGF), transforming growth factor-beta (TGF-β), platelet-derived angiogenesis growth factor (PDAF), transforming growth factor-beta (TGF-β), basic fibroblast growth factor (bFGF), vascular growth factor, vascular endothelial growth factor, and placental growth factor. These agents have been implicated in wound healing by increasing collagen secretion, vascular growth and fibroblast proliferation. Other exemplary non-limiting examples include Doxycycline, Tetracycline, peptides, proteins, hormones, DNA or RNA fragments, genes, cells, cell growth promoting compositions, and autologous platelet gel (APG). Alternatively, the bioactive or pharmaceutical agent can be coated on the outer surface of the liner. The agent or cell growth promoting factor on the outer surface of liner can activate cell growth and proliferation. Those cells adhere to the liner and anchor the liner securely to the vessel lumen and thus preventing migration. Moreover, tissue in-growth on the liner can also provide a seal around the junction of collateral arteries in the aneurysm and prevent endoleak.

According to the teaching of the present invention, the hardenable liner has a size no less than the aneurysm. As discussed above, it is important for the liner to conform to the inner surface of the aneurysm to avoid migration and endoleak. To achieve that, the liner has a size no less than the target aneurysm to conform to the inner surface of the aneurysm without stretching the liner excessively. Stretching of the liner may place excess stress on the aneurysm to be treated and may lead to a gap between the liner and aneurysm wall if the liner retracts after deploying in the aneurysm. However, folds or wrinkles may occur if the liner is larger than the target aneurysm. This may actually increase the thickness and strength of the liner further enhancing its ability to protect the aneurysm after the liner is transformed into the strengthening mode.

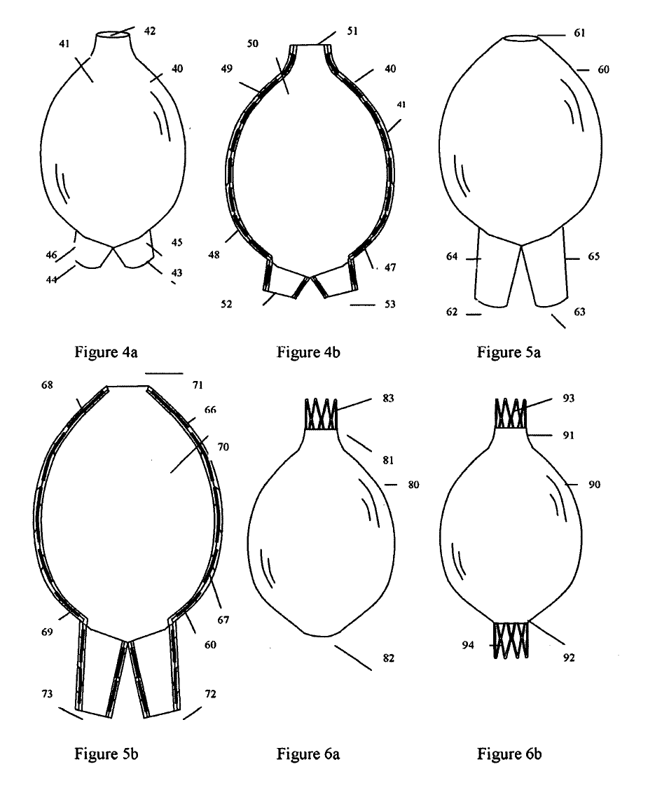

In another embodiment of the present invention, the liner is particularly suitable for lining an aneurysm disposed in close proximity to a bifurcation, such as an aortic aneurysm adjacent to the iliac artery. FIGS. 4a-b are the perspective and cross sectional views of the exemplary liners according to the teaching of this invention. In FIG. 4a, outer wall 40 of liner 41 is flexible with three openings 42, 43, 44. Two openings 43, 44 leading to the bifurcation are adjacent to each other. There are sleeves 45, 46 connected to openings 43, 44 respectively to enhance the seal between liner 41 and the vessel wall. The space between outer wall 40 and inner wall 47 comprises at least one chamber 48 filled by hardening agent 49 as depicted in the cross sectional view of liner 41 in FIG. 4b. Inner wall 47 defines blood flow conduit 50 with one inlet 51 and two outlets 52, 53. Each of the outlets 52, 53 leads to an iliac artery respectively. After the deployment within the aneurysm, liner 41 will have the shape defined by the morphology of the inner surface of the aneurysm wall. The shape of blood flow conduit 50 will be determined by the morphology of the inner surface of the aneurysm wall and the thickness of liner 41.

In yet another embodiment of the present invention, the liner is particularly suitable for lining aneurysm which has extended from aorta to iliac artery. FIGS. 5a-b are the perspective and cross sectional views of the exemplary liners according to the teaching of this invention. Liner 60 is hollow with three openings 61, 62, 63 as shown in FIG. 5a. Two of the openings 62, 63 leading to the bifurcation are adjacent to each other and are configured to mate with an iliac artery respectively. Sleeves 64, 65 extended from openings 62, 63 enhance the seal between liner 60 and the vessel wall and protect aneurysm in the iliac arteries. The space between outer wall 66 and inner wall 67 comprises at least one chamber 68 filled by hardening agent 69 as depicted in the cross sectional view of liner 60 in FIG. 5b. Inner wall 67 defines blood flow conduit 70 with one inlet 71 and two outlets 72, 73. After deployment within the aneurysm, blood flow conduit 70 will have a shape determined by both the inner surface of the aneurysm and the thickness of liner 60.

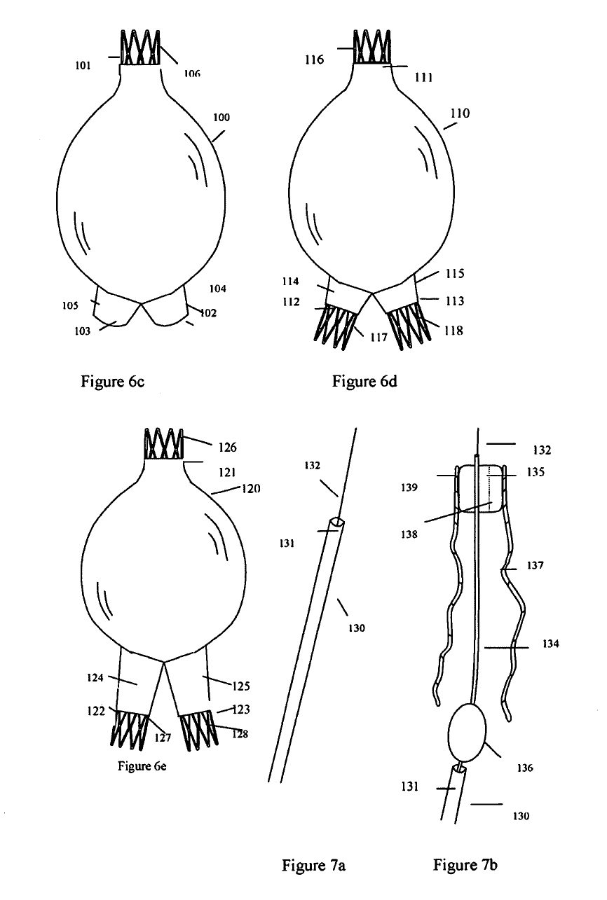

In yet another embodiment of the present invention, at least one stent is permanently fixed to one of the openings of the inflatable liner for anchoring and sealing the liner on the vessel wall. The stent is either self-expandable either or by the outward radial force exerted by another expandable element so that stent can expand and anchor liner to the vessel walls after deployment. Typical biocompatible materials for stent are stainless steel, Nitinol or plastic. FIGS. 6a-e are the perspective views of exemplary liners according to the teaching of this invention. As shown in FIG. 6a, liner 80 is hollow with two openings 81, 82. At least one stent 83 is permanently fixed to liner 80 near opening 81. Stent 83 is stitched, glued, or bonded to liner 80. Alternatively, liner 90 is hollow with two openings 91, 92 as illustrated in FIG. 6b. One stent 93 is permanently fixed to liner 90 near opening 91. Another stent 94 is permanently fixed to liner 90 near opening 92. Stents 93, 94 are stitched, glued, or bonded to liner 90. This embodiment of the present invention is particularly suitable for treating patients with Thoracic aortic aneurysm (TAA), aneurysms in the peripheral arteries, or abdominal aortic aneurysms (AAA) with some distance from the iliac bifurcation.

As shown in FIG. 6c, liner 100 is hollow with three openings 101, 102, 103. Two of the openings 102, 103 leading to the bifurcation have sleeve 104, 105 adjacent to each other. Stent 106 is permanently fixed to liner 100 near opening 101 by stitch, glue, or heat bonding. Alternatively, liner 110 is hollow with three openings 111, 112, 113 as illustrated in FIG. 6d. Two of the openings 112, 113 leading to the bifurcation have sleeve 114, 115 adjacent to each other. Stent 116 is permanently fixed to liner 110 near opening 111. One stent 117 is permanently fixed to sleeve 114 leading to one of the iliac arteries. Another stent 118 is permanently fixed to sleeve 115 leading to the other iliac artery. This embodiment of the present invention is particularly suitable for treating patients with aneurysms adjacent to bifurcation.

Liner 120 is hollow with three openings 121, 122, 123 as shown in FIG. 6e. Two of the openings 122, 123 leading to the bifurcation have sleeves 124, 125 adjacent to each other. Each of the openings 122, 123 is configured to mate with an iliac artery respectively. Sleeves 124, 125 extended from the openings 122, 123 enhance the seal between the liner 120 and the vessel wall and protect aneurysm in the iliac arteries. Stent 126 is permanently fixed to liner 120 near opening 121. Stents 127, 128 are stitched, glued, or bonded to sleeves 124, 125 leading to iliac arteries respectively. This embodiment of the present invention is particularly suitable for treating patients with aneurysms extended from aorta to iliac artery.

As discussed before, the aneurysm is usually weak and prone to rupture, it is important to be able to monitor the progress of expansion to ensure conformation to the aneurysm surface and avoid excess stress on the aneurysm. Radiopaque markers are placed on the surface or between walls of the liner. Alternatively, the status of expansion can be observed if the liner becomes radiopaque with additional radiopaque agent in it. This embodiment of the present invention provides physicians a safe tool to know directly the status of the expansion without guessing.

In the practice, physician needs to determine the appropriate liner to use for each patient. With the imaging techniques such as CT scan or MRI, the size and length of the patient's aneurysm can be measured accurately. Then, the physician can select the hardenable liner that best fit the patient's aneurysm anatomy. It is preferable to use a liner with outer diameter no less than the largest inner diameter of the aneurysm. Because of its flexibility in the pliable mode, the liner will conform to the inner wall of the aneurysm by a hemodynamic force with no gap for potential endoleak.

In one embodiment according to the present invention, a hardenable liner is pre-loaded into a delivery catheter such as what depicted in FIGS. 7a. Delivery catheter 130 has a retractable sheath 131 with compressed liner (not shown) in it. Guidewire 132 can pass through a lumen (not shown) in delivery catheter 130 and is used to direct delivery catheter 130 in patients' body. Within the lumen of delivery catheter 130 is a multilumen catheter 134, as shown in FIG. 7b. Multilumen catheter 134 has a lumen for guide wire 132, and lumens for delivery of saline for inflating distal balloon 135, proximal balloon 136. An expandable element, such as distal balloon 135, is positioned at the distal end of the multilumen catheter 134 to anchor liner 137 during the deployment. Various types of expandable elements, such as self-expandable stent, wire, mesh, balloon, etc. can be used in this invention. An inflatable balloon is used herein as an example. Inflatable distal balloon 135 is preferable to have an annular shape with a lumen 138 allowing blood flow through balloon 135 after inflation. In the delivery mode, portion of liner 137 near inlet 139 is mounted on top of balloon 135 with the inner surface against balloon 135. Optionally, a second expandable element, a proximal balloon 136 herein, is placed near the proximal end of multilumen catheter 134. After liner 137 and balloons 135, 136 are collapsed into the low profile configurations; they are radially compressed to fill a liner chamber (not shown) in the distal end of catheter 130. The liner 137 is covered with retractable sheath 131 and sterilized with various known sterilization methods.

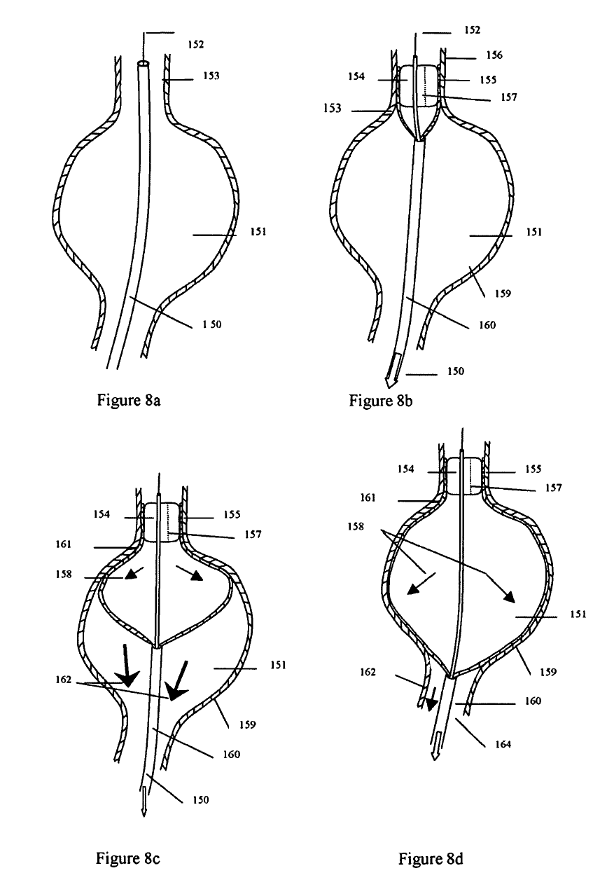

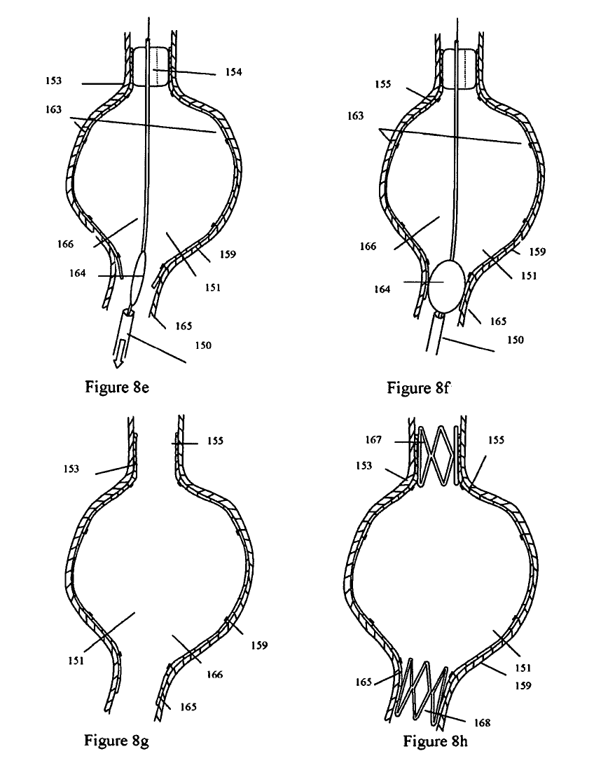

For a preferred deployment method of this invention, a multi-lumen balloon catheter 150 is used to deliver the liner in aneurysm 151 via the iliac artery using a minimally invasive technique. An inflatable liner with two openings (as shown in FIG. 1a) is used herein as an example to line aneurysm 151. As shown in FIG. 8a, delivery catheter 150 is guided by guidewire 152 and positioned in the aneurysm 151 with its distal end close to neck 153 of aneurysm 151. It is preferable that distal balloon 154 is deployed near neck 153 of aneurysm 151 to ensure that no excess stress is exerted upon aneurysm 151 as illustrated in FIG. 8b. After distal balloon 154 is inflated, a portion of liner 155 is pressed against vessel wall 156 by the inflated distal balloon 154. At the same time, blood flows through lumen 157 within distal balloon 154 (as indicated by arrow 158) to expand liner 155 radially toward aneurysm wall 159. As sheath 160 is retrieved to expose liner 155 in sheath 160, the expansion continues until outer wall 161 of liner 155 is against aneurysm wall 159 of aneurysm 151 as depicted in FIGS. 8c-d. As indicated by arrows 162 in FIGS. 8c-d, the existing blood in aneurysm 151 escapes from aneurysm 151 through a gap between catheter 150 and aneurysm wall 159. This procedure is safe because the hemodynamic pressure to expand liner 155 is the same hemodynamic pressure that existed in aneurysm 151 before treatment. No additional stress is placed on aneurysm wall 159 during the liner expansion. As liner 155 is expanded, the status of expansion is monitored by radiopaque markers 163 on the surface of liner 155 as illustrated in FIGS. 8e-f. Alternatively, the status of expansion can be observed if hardening agent (not shown) in liner 155 becomes radiopaque when additional radiopaque agent has been added to it. After aneurysm wall 159 has been completely covered by liner 155, a proximal balloon 164 is inflated at junction 165 between liner 155 and aneurysm wall 159 as shown in FIG. 8f. Proximal balloon 164 is also preferably of an annular shape and can be on the same catheter 150 or on a separate catheter. Proximal balloon 164 is to ensure the patency of blood flow conduit 166 at junction 165 during the expansion of liner 155. The activation of hardening agent (not shown) and the strengthening of liner 155 give additional strength to liner 155 and protects aneurysm wall 159 as shown in FIG. 8f. Finally, balloons 154 and 164 are collapsed, and delivery catheter 150 is retrieved from the patient's body leaving liner 155 in aneurysm 151 as shown in FIGS. 8g. Optionally, stents 167, 168 or, alternatively, membrane covered stents are placed at neck 153 and junction 165 respectively to ensure adequate seal as shown in FIG. 8h.

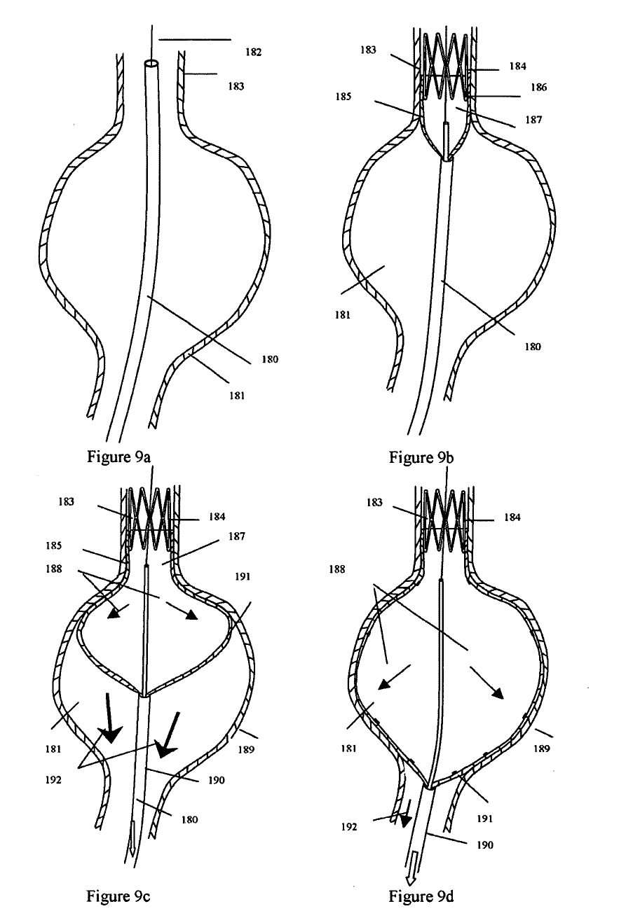

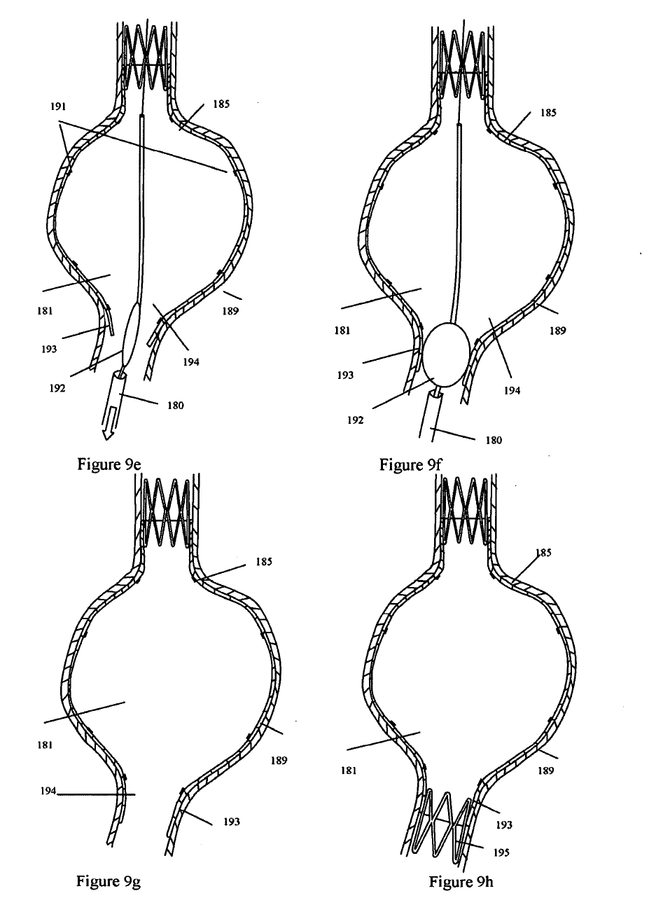

For another preferred deployment method of this invention, a multi-lumen catheter 180 is used to deliver a stent attached liner in the aneurysm 181 via the iliac artery with minimum invasivity. A liner with a self expandable stent affixed to one of its openings (as shown in FIG. 6a) is used herein as an example to line aneurysm 181. A balloon expandable stent can also be used. As shown in FIG. 9a, delivery catheter 180 is guided by guidewire 182 and positioned in aneurysm 181 with its distal end close to neck 183 of aneurysm 181. It is preferable that distal stent 184 is deployed near neck 183 of aneurysm 181 to ensure that no excess stress is exerted upon aneurysm 181 as illustrated in FIG. 9b. After distal stent 184 is deployed, a portion of liner 185 is pressed against vessel wall 186 by the deployed stent 184. At the same time, blood flows through lumen 187 in distal stent 184, as indicated by arrows 188, in order to expand liner 185 radially toward aneurysm wall 189 as depicted in FIGS. 9c-d. As sheath 190 is retrieved to expose liner 185 in sheath 190, the expansion continues until outer wall 191 of liner 185 is against aneurysm wall 189 of aneurysm 181. As indicated by arrows 192 in FIGS. 9c-d, the existing blood in aneurysm 181 escapes from aneurysm 181 through the gap between catheter 180 and aneurysm wall 189. This procedure is safe because the hemodynamic pressure to expand liner 185 is the same hemodynamic pressure that existed in aneurysm 181 before treatment. No additional stress is placed on aneurysm wall 189 during the liner expansion. As liner 185 is expanding, the status of expansion is monitored by radiopaque markers 191 on the surface of liner 185 as shown in FIG. 9e. Alternatively, the status of expansion can be observed if hardening agent (not shown) becomes radiopaque when additional radiopaque agent has been added to it. After aneurysm wall 189 has been completely covered by liner 185, a proximal balloon 192 is inflated at junction 193 between liner 185 and aneurysm wall 189 as shown in FIGS. 9e-f. Proximal balloon 192 is preferably on the same catheter 180 or on a separate catheter. Proximal balloon 192 is to ensure the patency of blood flow conduit 194 at junction 193 during the expansion of liner 185. The activation of hardening agent (not shown) gives additional strength to liner 185 and protects aneurysm wall 189 as shown in FIG. 9f. After the transition has completed, balloon 192 is collapsed, and delivery catheter 180 is retrieved from the patient's body leaving liner 185 in aneurysm 181 as shown in FIGS. 9g. Optionally, stent 195 or, alternatively, membrane covered stent is placed at junction 193 to ensure adequate seal as shown in FIG. 9h.

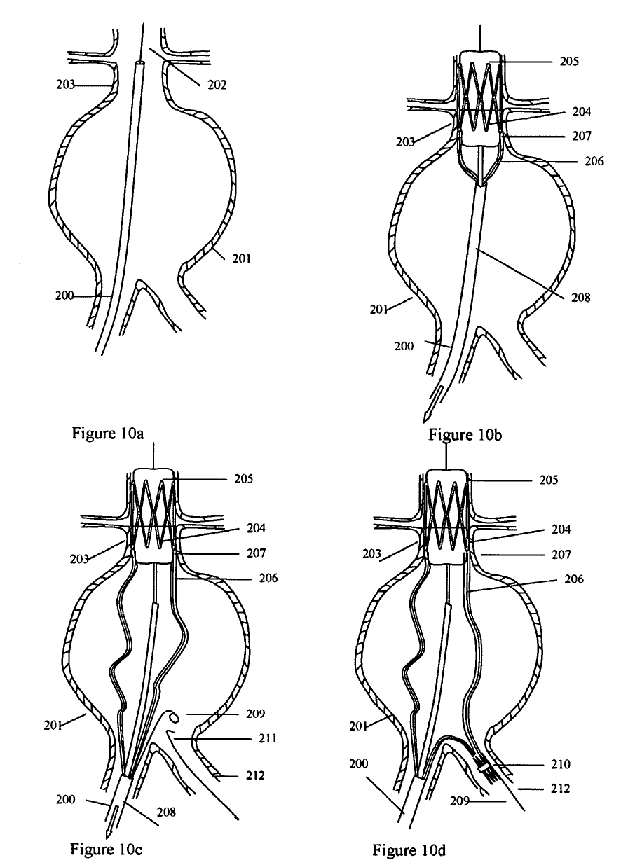

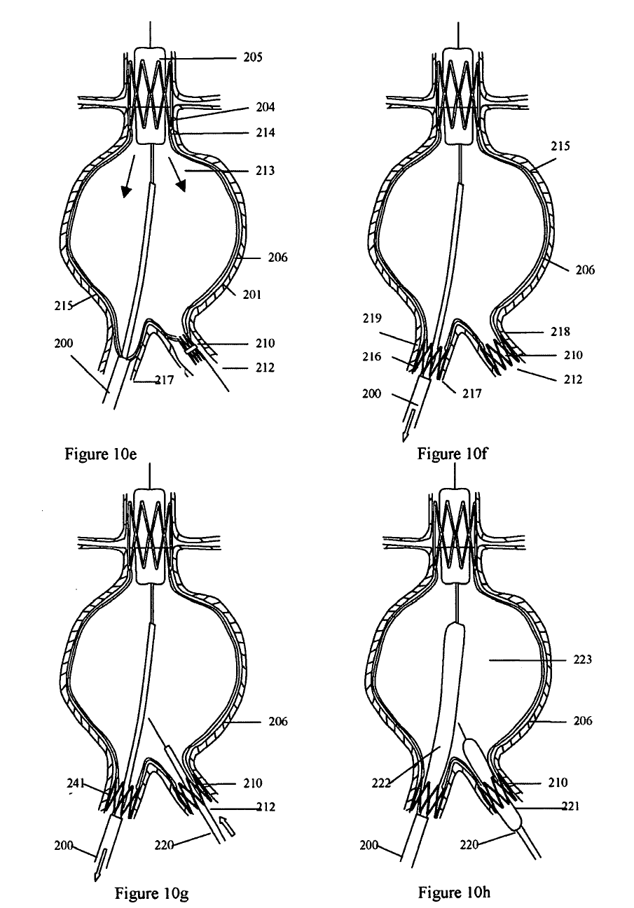

For yet another preferred deployment method of this invention, multi-lumen delivery catheter 200 is used to deliver the stent attached liner in aneurysm 201 via the iliac artery with minimum invasivity. A liner with three stents affixed to its three openings (as shown in FIG. 6d) is used herein as an example to line aneurysm 201 close to the bifurcation. Other exemplary stent attached liners can also be deployed with this method. As shown in FIG. 10a, delivery catheter 200 is guided by guidewire 202 and positioned in aneurysm 201 with its distal end close to neck 203 of aneurysm 201. It is preferable that distal stent 204 is deployed by a distal balloon 205 near neck 203 of aneurysm 201 to ensure that no excess stress is exerted upon aneurysm 201 as illustrated in FIG. 10b. A balloon expandable stent 204 is used herein as an example. Other types of stent such as self expandable stent can also be used in this invention. After distal stent 204 is deployed, a portion of liner 206 is pressed against vessel wall 207 by the deployed stent 204. Then, sheath 208 of catheter 200 is removed to expose the to-be expanded liner 206 and a wire 209 linked to an iliac stent 210 as illustrated in FIG. 10c. Simultaneously, a wire 211 is inserted in aneurysm 201 via left iliac artery 212 to pull wire 209 and iliac stent 210 to the left iliac artery 212 for deployment as shown in FIG. 10d. Distal balloon 205 is then deflated slightly allowing blood flow (as indicated by arrows 213) through space 214 between balloon 205 and distal stent 204 to expand liner 206. Under this hemodynamic pressure, liner 206 expands radially toward aneurysm wall 215 and eventually conforms to the inner surface of aneurysm wall 215 of aneurysm 201 as depicted in FIGS. 10e. This procedure is safe because the hemodynamic force to expand liner 206 is no larger than the hemodynamic force before the procedure. No additional stress is placed on aneurysm wall 215 during the expansion of liner 206.

After aneurysm wall 215 is completely covered by liner 206, both iliac stents 210, 216 are deployed in iliac arteries 212, 217 respectively as shown in FIG. 10f. They are used to ensure seal at junctions 218, 219 between liner 206 and iliac arteries 212, 217. Self expandable stents 210, 216 are used herein as an example. Other types of stents such as balloon expandable stents can also be used in this invention. As shown in FIG. 10g, a balloon catheter 220 is inserted in liner 206 via left iliac artery 212. Once it is in position, balloon 221 on the distal end of catheter 220 is inflated with saline. As shown in FIG. 10h, a proximal balloon 222 on delivery catheter 200 is also inflated by saline. Both balloons 221, 222 are used to ensure patency of flow conduit 223 when liner 206 is hardened. The activation of hardening agent (not shown) in liner 206 gives additional strength to liner 206 and protects aneurysm wall 215 as shown in FIG. 10h. Finally, all balloons 205, 221, 222 are deflated. Both delivery catheter 200 and balloon catheter 220 are retrieved from the patient's body leaving liner 206 in aneurysm 201 as shown in FIG. 10i. This invention is particularly suitable for treating patients with abdominal aortic aneurysms near the iliac bifurcation.

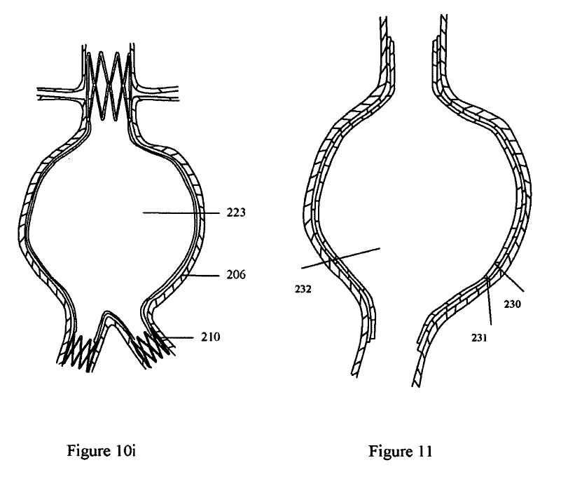

In another embodiment of this invention, a plurality of hardenable liners can be used to treat the aneurysm. As shown in FIG. 11, two liners 230, 231 are used to line and protect the aneurysm 232. Liners 230, 231 form a composite and enhance the overall strength of liners 230, 231 thus increasing their ability to reduce the hemodynamic pressure on the aneurysm wall 232. Individual liner can be deployed in the aneurysm sequentially following the methods described in this invention. Or a hardenable liner can comprise more than one layer. Compared with the liner of multiple layers, deploying the liner one layer at a time can reduce the size of the delivery catheter therefore enhancing its ability to maneuver through patient's tortuous iliac anatomy. As a result, each liner can be introduced in the aneurysm with a minimum invasivity. Alternatively, hardenable liners of various flexibility in the pliable mode can be used in this invention. For example, the liner with a higher flexibility can be used as the liner adjacent to the inner surface of aneurysm for the optimum conformation to the aneurysm to prevent endoleak. Alternatively, hardenable liners of various stiffness in the strengthening mode can be used in this invention. For example, the liner with a higher stiffness can be used as the liner forming the flow conduit for the enhanced support for the conduit. Alternatively, hardenable liners of various compositions can be used in this invention. For example, the liner with a higher bioactive agent content can be used as the liner adjacent to the inner surface of aneurysm for the optimum efficacy. Alternatively, hardenable liners of various configurations can be used in this invention. For example, the liner with a higher surface roughness can be used as the liner adjacent to the inner surface of aneurysm for the enhanced ability to promote tissue in-growth and fixation.

There are several benefits for this present invention to treat aneurysm. First, the liner can strengthen the aneurysm wall and prevent the rupture of aneurysm by reducing the hemodynamic pressure on the aneurysm wall. Second, the collapsed liner is flexible so that it can be easily loaded in a catheter and access the aneurysm site via iliac artery and then deployed in the aneurysm with minimum invasivity. Third, the flexibility of the liner and the hemodynamic force allow the liner to conform to the inner surface of the aneurysm wall without gap between them. After the hardening agent is activated and the liner is stiffened, the liner will be “locked” in the aneurysm without endoleak or migration. Fourth, less material is required to cover the inner surface of aneurysm wall than filling the whole aneurysm. The resulting liner is more flexible than the filler structure that fills the whole aneurysm. This flexible liner is more compatible with the body movement and adjacent organs. Fifth, there is no excess amount of stress on the aneurysm wall during the expansion of the liner. In order to prevent endoleak and migration, it is essential to have close contact between the outer wall of the liner and the surface of the aneurysm wall. To achieve that, the whole aneurysm (other than the tubular flow conduit within the aneurysm) needs to be filled as what was disclosed in the prior arts. In the present invention, the close contact between the aneurysm wall and the outer wall of the liner is a result of flexible walls and the hemodynamic force. It is not necessary to fill the whole aneurysm in order to close the gap between the aneurysm wall and the liner. As a result, the systems and methods provided by this present invention are safer than what were disclosed in the prior arts. Sixth, the present invention can enhance the adhesion of the liner to the aneurysm wall to further reduce the risk of liner migration and endoleak. Seventh, this invention enables the use of bioactive or pharmaceutical agents in the liner to treat aneurysm without dilution. The pathological processes involved in aneurysm progression can be stabilized and possibly be reversed. Eighth, the flexible liner does not have the issue of kinking or occlusion of blood flow which is common in tubular stent graft. Ninth, the durability of the liner is better than the tubular stent graft because there is no untreated space, which is prone to endoleak, between the liner and aneurysm wall.

Those skilled in the art will further appreciate that the embodiments according to the teaching of present invention may include other specific forms or characteristics without departing from the spirit of this invention thereof. The present invention is not limited in the particular embodiments described in detail therein. The foregoing description discloses only exemplary embodiments, other variations are considered as being within the scope of the present invention. Numerous references cited herein are incorporated by reference in their entirety.

I claim:

1. A system to protect the wall of an aneurysm in a vessel wherein the system comprises:
a liner comprising one or more chambers, said one or more chambers comprising hardening agent, wherein said liner is configured to conform to the interior surface of the aneurysm following introduction of said liner into the vessel, and wherein said hardening agent being activated by body environment to become stiff.

2. The system as set forth in claim 1 further comprising means for anchoring said liner to the interior of the vessel.

3. The system as set forth in claim 2, wherein said means for anchoring said liner comprises one or more expandable elements coupled to said liner.

4. The system as set forth in claim 3, wherein said one or more expandable elements comprises a stent.

5. The system as set forth in claim 1, wherein said liner comprises an inner wall defining a main flow conduit of the vessel proximate the aneurysm following introduction of the liner into the vessel, said conduit comprising an inlet and one or more outlets.

6. The system as set forth in claim 5, wherein said inner wall is permeable to body fluid.

7. The system as set forth in claim 1, wherein the hardening agent is comprised of a moisture-cured resin, wherein said moisture-cured resin is selected from the group consisting of silicone, cyanoacrylate, acrylate, polyurethane.

8. The system as set forth in claim 1, wherein the hardening agent is comprised of a calcium compound.

9. The system as set forth in claim 8, wherein said calcium compound is selected from the group consisting of dicalcium phosphate anhydrous, CaCO3, Ca(OH)2, alpha-tricalcium phosphate, tetracalcium phosphate, amorphous calcium phosphate, monocalcium phosphate monohydrate, monocalcium phosphate anhydrous, dicalcium phosphate dehydrate, beta-tricalcium phosphate, octacalcium phosphate, and mixture thereof.

10. The system as set forth in claim 1, wherein the hardening agent is comprised of a polymer solution.

11. The system as set forth in claim 10, wherein the polymer solution is comprised of a biocompatible polymer and a biocompatible solvent.

12. The system as set forth in claim 11, wherein the biocompatible solvent is selected from the group consisting of ethanol, dimethylsulfoxide, ethyl lactate, and acetone.

13. The system as set forth in claim 11, wherein the biocompatible polymer is selected from the group consisting of cellulose acetate, cellulose acetate butyrate, cellulose diacetate, nitrocellulose, polyurethane, polycarbonate, polyester, ethylene vinyl alcohol, ethylene vinyl acetate, polyacrylonitrile, polyvinyl acetate, polyvinyl alcohol, acrylics, and copolymer and mixture thereof.

14. The system as set forth in claim 1 wherein said hardening agent comprises a bioactive or a pharmaceutical active component.

15. The system as set forth in claim 1, wherein the liner comprises an outer surface comprising a bioactive or a pharmaceutical active component.

16. The system as set forth in claim 1, wherein the liner comprises an outer surface and surface area, wherein said outer surface is treated with fibers, fibril, foam, or roughening to increase the surface area.

17. The system as set forth in claim 1 further comprising means to introduce a hemodynamic force in said liner whereby said liner expands and conforms to the interior surface of the aneurysm.

18. An endovascular system to protect aneurysm wall comprising:
a hardenable liner having an inner wall and an outer wall and a hardening agent encapsulated between walls, said hardenable liner having a first configuration and a second configuration, said first configuration being low profile delivery configuration and insertable into blood vessel, said second configuration being defined by the blood vessel, said hardenable liner being expandable plastically from said first configuration to said second configuration by the hemodynamic force in said blood vessel, said hardenable liner having at least one wall being permeable to moisture, said hardening agent being activated by said moisture to become stiff.

19. A method of treatment of an aneurysm comprising:
providing an hardenable liner comprising one or more chambers and hardening agent; and
anchoring a portion of the hardenable liner in the vessel adjacent the aneurysm with a first expandable element; and
introducing hemodynamic force in the hardenable liner whereby said liner expands and conforms to the interior surface of the aneurysm; and
providing and deploying a second expandable element to open the flow conduit at the junction between said hardenable liner and the vessel; and
introducing body fluid into the one or more chambers whereby said hardening agent is stiffened and protects the vessel.
